(19) United States
(12) Patent Application Publication
(10) Pub. No.: US 2011/0151394 A1
(43) Pub. Date: Jun. 23, 2011

(54) PLAQUE TOOTHTOOL AND DENTIFRICE SYSTEM

(76) Inventors: Ioana M. Rizoiu, San Clemente, CA (US); Dmitri Boutoussov, Dana Point, CA (US); David M. Mulder, Foothill Ranch, CA (US)

(21) Appl. No.: 12/972,180

(22) Filed: Dec. 17, 2010

Related U.S. Application Data
(60) Provisional application No. 61/308,290, filed on Feb. 25, 2010.
(60) Provisional application No. 61/287,497, filed on Dec. 17, 2009.

(51) Int. Cl. A61B 6/14

(52) U.S. Cl. 433/29

(57) ABSTRACT

A disclosing system for teeth having an electromagnetic radiation emitting toothtool and a dentifrice with a photosensitive agent is disclosed.

### PRIORITY AND RELATED-APPLICATION INFORMATION

This application claims the benefit of U.S. Provisional Application No. 61/308,290 (Att. Docket BI9068CIP2PR), filed Feb. 25, 2010 and entitled PLAQUE TOOTHTOOL AND DENTIFRICE SYSTEM, and U.S. Provisional Application No. 61/287,497 (Att. Docket BI8034PR), filed Dec. 17, 2009 and entitled LIGHTING DEVICE AND MOUTHWASH ORAL AGENT DISCLOSING COMBINATION, the entire contents of both which are hereby incorporated by reference.

U.S. Provisional Application No. 61/308,290 is a continuation-in-part of U.S. application Ser. No. 11/890,047 (Att. Docket BI9068CON2), filed Aug. 3, 2007 and entitled ELECTROMAGNETIC RADIATION EMITTING TOOTHBRUSH AND DENTIFRICE SYSTEM, now abandoned, which is a continuation of U.S. application Ser. No. 10/624,963 (Att. Docket BI9068CON), filed Jul. 21, 2003 and entitled ELECTROMAGNETIC RADIATION EMITTING TOOTHBRUSH AND DENTIFRICE SYSTEM, now U.S. Pat. No. 7,261,558, which is a continuation of U.S. application Ser. No. 09/445,947 (Att. Docket BI9068P), filed Aug. 29, 2000 and entitled ELECTROMAGNETIC RADIATION EMITTING TOOTHBRUSH AND DENTIFRICE SYSTEM, now U.S. Pat. No. 6,616,451, which was filed as Application No. PCT/US98/12836 on Jun. 19, 1998, which claims the benefit of U.S. Provisional Application No. 60/050,343 (Att. Docket BI9068PR), filed Jun. 20, 1997 and entitled ELECTROMAGNETIC RADIATION EMITTING TOOTHBRUSH AND TRANSPARENT TOOTHPASTE COMBINATION, the contents all of which are expressly incorporated herein by reference. U.S. Provisional Application No. 61/308,290 is also a continuation-in-part of U.S. application Ser. No. 11/438,091 (Att. Docket BI9887P), filed May 18, 2006 and entitled ELECTROMAGNETIC RADIATION EMITTING TOOTHBRUSH AND DENTIFRICE SYSTEM, now U.S. Pat. No. 7,467,946, which claims the benefit of U.S. Provisional Application No. 60/682,752 (Att. Docket BI9068CIPPR), filed May 18, 2005 and entitled ELECTROMAGNETIC RADIATION EMITTING TOOTHBRUSH AND DENTIFRICE SYSTEM, and U.S. Provisional Application No. 60/688,109 (Att. Docket BI9887PR), filed Jun. 6, 2005 and entitled ELECTROMAGNETIC RADIATION EMITTING TOOTHBRUSH AND DENTIFRICE SYSTEM, and U.S. Provisional Application No. 60/739,314 (Att. Docket BI9893PR), filed Nov. 23, 2005 and entitled ELECTROMAGNETIC RADIATION EMITTING TOOTHBRUSH AND DENTIFRICE SYSTEM, the entire contents of all which are hereby incorporated by reference. U.S. Provisional Application No. 61/308,290 is related to U.S. application Ser. No. 11/074,452 (Att. Docket BI9546CON), filed Mar. 8, 2005 and entitled RADIATION EMITTING APPARATUS WITH SPATIALLY CONTROLLABLE OUTPUT ENERGY DISTRIBUTIONS, now U.S. Pat. No. 7,270,657, which is a continuation of U.S. application Ser. No. 10/229,374 (Att. Docket BI9546P), filed Aug. 26, 2002 and entitled RADIATION EMITTING APPARATUS WITH SPATIALLY CONTROLLABLE OUTPUT ENERGY DISTRIBUTIONS, now U.S. Pat. No. 6,942,658, the entire contents of both which are hereby incorporated by reference. U.S. Provisional Application No. 61/308,290 also is related to U.S. Pat. No. 6,616,447 (Att. Docket BI9322P), which issued on Sep. 9, 2003 and which claims the benefit of U.S. Provisional Application No. 60/249,015 (Att. Docket BI9322PR), filed Nov. 15, 2000 and entitled DEVICE FOR DENTAL CARE AND WHITENING, the entire contents of both which are hereby incorporated by reference.

U.S. Provisional Application No. 61/287,497 is a continuation-in-part of U.S. application Ser. No. 11/438,091 (Att. Docket BI9887P), filed May 18, 2006 and entitled ELECTROMAGNETIC RADIATION EMITTING TOOTHBRUSH AND DENTRIFICE SYSTEM, issued as U.S. Pat. No. 7,467,946, which claims the benefit of U.S. Provisional Application No. 60/739,314 (Att. Docket BI9893PR), filed Nov. 23, 2005 and entitled ELECTROMAGNETIC RADIATION EMITTING TOOTHBRUSH AND DENTRIFICE SYSTEM, and U.S. Provisional Application No. 60/688,109 (Att. Docket BI9887PR), filed Jun. 6, 2005 and entitled ELECTROMAGNETIC RADIATION EMITTING TOOTHBRUSH AND DENTRIFICE SYSTEM, and U.S. Provisional Application No. 60/682,752 (Att. Docket BI9068CIPPR), filed May 18, 2005 and entitled ELECTROMAGNETIC RADIATION EMITTING TOOTHBRUSH AND DENTRIFICE SYSTEM, the contents all of which are expressly incorporated herein by reference. U.S. Provisional Application No. 61/287,497 is also a continuation-in-part of U.S. application Ser. No. 12/631,642 (Att. Docket BI9914CIP), filed Dec. 4, 2009 and entitled FLUID CONDITIONING SYSTEM, which is a continuation-in-part of U.S. application Ser. No. 11/330,388 (Att. Docket BI9914P), filed Jan. 10, 2006 and entitled FLUID CONDITIONING SYSTEM, now abandoned, which claims the benefit of U.S. Provisional Application No. 60/709,714 (Att. Docket BI9914PR), filed Aug. 19, 2005 and entitled FLUID CONDITIONING SYSTEM, and U.S. Provisional Application No. 60/696,475 (Att. Docket BI9903PR), filed Jul. 1, 2005 and entitled FLUID CONDITIONING SYSTEM, and U.S. Provisional Application No. 60/645,427 (Att. Docket BI9694CIPPR), filed Jan. 19, 2005 and entitled FLUID CONDITIONING SYSTEM, the contents all of which are expressly incorporated herein by reference. U.S. application Ser. No. 12/631,642 is also a continuation-in-part of U.S. application Ser. No. 11/033,044 (Att. Docket BI9694P), filed Jan. 10, 2005 and entitled FLUID CONDITIONING SYSTEM, now abandoned, which claims the benefit of U.S. Provisional Application No. 60/535,110 (Att. Docket BI9696PR), filed Jan. 8, 2004 and entitled FLUID CONDITIONING SYSTEM, the entire contents all of which are expressly incorporated herein by reference.

### BACKGROUND OF THE INVENTION

1. Field of the Invention

This invention relates generally to dental hygiene and, more particularly, to plaque identifying device and compound combinations.

2. Description of Related Art

Proper dental hygiene is not only important for dental health reasons, but for personal appearance considerations as well. One of the many techniques people employ to improve their personal appearance through dental hygiene is to have their teeth whitened. Teeth whitening techniques vary in cost, convenience, and effectiveness. Dental health professionals use many techniques to whiten teeth to improve their patients' personal appearance. High pressure baking soda solutions are used to scour teeth in a process analogous to sand-blasting. Chemical treatments abound for bleaching the teeth. However, these treatments require the inconvenience of visiting a dental health practice and the expense involved in having the treatment performed by a dental health professional.

Brushing with a dentifrice, whether a toothpaste, gel, cream, or powder, has some effect in whitening teeth due to the abrasive action of the dentifrice on the teeth. A relatively simple method of whitening teeth comprises brushing them with a dentifrice comprising whitening agents. Hydrogen peroxide, a common whitening agent, is a component in many off-the-shelf toothpastes and tooth gels. However, certain diets and activities, such as smoking, stain the teeth beyond the ability of a dentifrice alone to sufficiently whiten them.

The prior art discloses toothbrushes equipped with a light source that can illuminate the brushing region and provide beneficial radiation to the tooth surface. Examples of illuminating toothbrushes are disclosed in U.S. Pat. Nos. 5,306,143 entitled DENTAL HYGIENE APPLIANCE; 5,160,194 entitled TOOTHBRUSH WITH EXTERNALLY ILLUMINATED BRISTLES; 5,030,090 entitled OPTICAL TOOTHBRUSH AND METHOD OF USE; and 4,779,173 entitled ILLUMINATED BRUSH DEVICE, all of which are expressed incorporated herein by reference in their entireties.

Regardless of the care exercised and time spent in a brushing session, one or more of stains, tartar, calculus, plaque and/or bacteria (hereafter “undesired substance”) may not be dislodged and removed from the tooth surfaces.

### SUMMARY OF THE INVENTION

According to the invention, chemical products are provided in dentifrices, such as, but not limited to, mouthwashes, thereby causing the dentifrices to function as disclosing solutions to make or facilitate the making of such undesired substances visible.

For instance, chemical products can be selected to react with undesired substances in such a manner as to render the undesired substances visible in the presence of radiation from a hand held wand, e.g., a brushless toothbrush-like device). The device is coupled to or contains a radiation source having a certain wavelength (e.g., visible light). Special device heads can be coupled with appropriate radiating source(s), and, alternatively, or additionally, according to an aspect of the present invention, with no bristles, for this purpose.

Such chemical products can be provided as an ingredient of a special mouthwash that may be, but is not limited to being, clear, colorless, transparent to visible light, about entirely transparent to visible light, non-opaque, about entirely non-opaque, and/or of a translucency about the same as (or visually indistinguishable from) pure H2O. Such products can alternatively, but not equivalently, be provided as an ingredient of a special toothpaste, tooth gel, and/or tooth powder, and/or a special tooth gel, that is one or more of clear, colorless, transparent to visible light, about entirely transparent to visible light, not opaque, about entirely non-opaque, and/or having a translucency about the same as or visually indistinguishable from pure H2O.

In modified embodiments of the invention, the dentifrice is tinted. In one embodiment of the invention, the dentifrice is a clear mouthwash comprising at least about 1.5 percent hydrogen peroxide and a clear base. In one exemplary embodiment, the base comprises water, poloxamer 407, glycerine, flavor and sodium saccharin. In another exemplary embodiment, the base comprises fluoride. The clear dentifrice (e.g., mouthwash) maximizes transmission of radiation therethrough, to thereby maximize an interaction of the chemical products with the radiation throughout the thickness of the dentifrice.

Any of the above may or may not, in any combination, be supplied with, contacting and/or within, the device according to the invention.

In further accordance with the invention, use may be made of a chemical product which reacts with an undesired substance in such a manner that radiation of an appropriate wavelength will have the effect of revealing (e.g., to the naked eye) and/or removing the undesired substance, or of altering the nature of the undesired substance, as by softening it, in a manner to facilitate complete removal by a swishing, pressurized hydraulic, rinsing and/or brushing action.

The dentifrice is formulated in one embodiment to comprise materials that may include a photosensitive agent functional (e.g., when irradiated) to provide or enhance provision of an anti-bacterial, anti-caries, anti-gingivitis, anti-tartar or anti-plaque functionality. The photosensitive agent can be formulated to react to emitted radiation of the device during use to enhance identification and/or removal of the undesired substance. A removal enhancement provided by the dentifrice may be direct, such as when the photosensitive agent reacts to the radiation and, in turn, reacts with the undesired substance to remove it, and/or the photosensitive agent can comprise a disclosing agent that renders an undesired substance visible under radiation produced by the device by changing the color of the disclosing agent. The disclosing agent can have an affinity for the undesired substance resulting in the disclosing agent being concentrated about the undesired substance relative to other areas on the target surface. As a result, the irradiated disclosing agent announces locations of undesired substance on tooth surfaces (e.g., so that brushing can be performed until the user observes that an undesired substance has been removed from all visible tooth surfaces).

According to one exemplary embodiment of the invention, the chemical product consists of or contains Black Shade No. 4625, which can be obtained by Crompton & Knowles, Ingredient Technology Division, of Reading, Pa. In this embodiment, the device is coupled to or contains a radiation source that produces radiation at a wavelength, or wavelengths, of between 0.8 mm and 1 mm. As presently embodied, the radiation source produces polychromatic radiation of wavelengths ranging from 0.8 mm to 1 mm. In a modified embodiment of the invention, the radiation source produces polychromatic radiation of wavelengths comprising at least a portion of the different wavelengths in the 0.8 mm to 1 mm range. In still another modified embodiment, the radiation source produces radiation of a single wavelength in the 0.8 mm to 1 mm range. The radiation source may comprise, for example, a Nd:YAG laser.

The source is adjusted to emit radiation at an energy level selected on the basis of the expected concentration of the Black Shade No. 4625 in the material to be treated. The Black Shade No. 4625 will stain, for example, cariogenic bacteria or streptococcus faecalis black so that this bacteria, or any other undesired substance absorbing Black Shade No. 4625, will readily absorb the radiation. As an alternative to the Black Shade No. 4625 in the example, any other substance for staining the undesired substance, for example, black can be used. Other photosensitive substances may be used in addition to, or as an alternative to, black. Agents comprising oranges, reds, browns, yellows, greens, blues, etc., supplied, for example, by Crompton & Knowles, Ingredient Technology Division, of Reading, Pa., may be selected, along with radiation sources having corresponding wavelengths. According to one specific embodiment, the chemical product is sudan red as a vital stain when the radiation source is an argon laser.

The radiation energy density can be made sufficiently high to directly vaporize the stained substance. For example, in the case where streptococcus faecalis is stained with Black Shade No. 4625 and irradiated by an Nd:YAG laser, this effect can be achieved with an energy density of the order of 10 J/cm².

To improve efficiency in accordance with the present invention, the dentifrice transmits an optimum amount of radiation therethrough. In an embodiment of the invention, an optimum amount of transmission through the dentifrice comprises transmitting radiation at wavelengths and intensities thereof to facilitate reaction of the photosensitive agent, and substantially no additional radiation beyond that. Embodiments of the invention have the photosensitive agents dispersed throughout the dentifrice.

During use, the dentifrice is dispersed in varying thicknesses over the target surface, which comprises the teeth and gums. To be effective, the radiation should penetrate through the dentifrice's varying thickness, so that significant portions of the photosensitive agent throughout the varying thicknesses are irradiated and react. For this to occur, the dentifrice transmits the radiation through the varying thicknesses, thereby enabling the significant portions of the dispersed photosensitive agent throughout the dentifrice to substantially absorb the radiation and react

Also, an identifying system for teeth having an electromagnetic radiation emitting hand-held oral device (e.g., toothbrush or toothtool) and a dentifrice with a photosensitive agent is disclosed. The toothtool has a target-facing surface (e.g., an emitting structure such as one comprising lightguides, and/or a target-facing, e.g., cleaning, surface such as bristles). The toothtool is adapted to direct electromagnetic radiation toward the target-facing surface. The electromagnetic radiation may be monochromatic or polychromatic. Further, the electromagnetic radiation may be blue or substantially blue, and may be substantially free of ultraviolet radiation. The photosensitive agent is dispersed throughout the dentifrice. The dentifrice transmits the electromagnetic radiation through a varying thickness of dentifrice disposed over a target surface during use of the system. As a result, a significant portion of the photosensitive agent reacts, resulting in disclosing undesirable substances and/or foaming. The dentifrice may be opaque, clear and/or may have clear abrasive particles. The dentifrice typically has an orange appearance and/or comprises clear abrasive particles. Alternatively, the dentifrice can have a yellow appearance and/or comprise clear abrasive particles. In another implementation, the dentifrice has a yellow-orange appearance and/or comprises clear abrasive particles. In other embodiments, the dentifrice can alternatively or additionally comprise agents and/or properties selected from one or more of, but not limited to, being clear, being non-clear (e.g., opaque), being a gel, being a paste, being a liquid or non-viscous fluid, being orange, being yellow, being orange-yellow, including an agent reactive or highly reactive or most reactive to (e.g., having an isosbestic point in the color) violet, blue, and/or green stimulation light to cause caries to fluoresce blue, green, and/or yellow, (e.g., respectively), being configured to cause caries to fluoresce blue (e.g., rather than green), being configured to cause caries to fluoresce blue-green (e.g., rather than just green), including an agent reactive or highly reactive or most reactive to (e.g., having an isosbestic point in the color) blue, green, and/or yellow stimulation light to cause caries to fluoresce green, yellow, and/or orange (e.g., respectively), having clear particles, having non-clear (e.g., opaque) particles, having abrasive particles, having non-abrasive particles, having orange particles, having yellow particles, having orange-yellow particles, and/or having particles that react to electromagnetic energy (e.g., that are photosensitive or photoactive) in any way described herein with reference to the dentifrice.

In an aspect of the invention, the teeth operating system comprises a dentifrice that has a photosensitive agent that reacts substantially only to electromagnetic radiation within a predetermined range (e.g., blue wavelengths). The teeth operating system further comprises a toothtool having a target-facing surface, the toothtool being adapted to direct electromagnetic radiation toward the target-facing surface, wherein the electromagnetic radiation is bound to wavelengths that are substantially within the predetermined range.

In a further embodiment of the invention, the photosensitive agent has an orange colored agent with a relatively high reaction rate when exposed to electromagnetic radiation in a highly reactive portion of the predetermined range compared to an average reaction rate when the photosensitive agent is exposed to electromagnetic radiation over the predetermined range. In another embodiment of the invention, the photosensitive agent has a yellow colored agent with a relatively high reaction rate when exposed to electromagnetic radiation in a highly reactive portion of the predetermined range compared to an average reaction rate when the photosensitive agent is exposed to electromagnetic radiation over the predetermined range. In a further embodiment of the invention, the photosensitive agent has an orange-yellow colored agent with a relatively high reaction rate when exposed to electromagnetic radiation in a highly reactive portion of the predetermined range compared to an average reaction rate when the photosensitive agent is exposed to electromagnetic radiation over the predetermined range. Additionally, the polychromatic electromagnetic radiation emitted by the toothtool is bound to wavelengths that are substantially within the highly reactive portion of the predetermined range.

In an aspect of the invention, a method of tuning the operating system for teeth includes a step of formulating the dentifrice with photosensitive agents that react to a range of electromagnetic radiation, and a step of providing a toothtool that emits that electromagnetic radiation range.

In another aspect of the invention, a method of tuning the operating system includes a step of designing a toothtool that emits a range of electromagnetic radiation (e.g., of blue wavelengths) and another step of formulating a dentifrice with a photosensitive agent (e.g., of a yellow-colored and/or orange-colored agent or dye) that reacts to energy within the electromagnetic radiation range.

While the apparatus and method has or will be described for the sake of grammatical fluidity with functional explanations, it is to be expressly understood that the claims, unless indicated otherwise, are not to be construed as limited in any way by the construction of “means” or “steps” limitations, but are to be accorded the full scope of the meaning and equivalents of the definition provided by the claims under the judicial doctrine of equivalents.

Any feature or combination of features described or referenced herein are included within the scope of the present invention provided that the features included in any such combination are not mutually inconsistent as will be apparent from the context, this specification, and the knowledge of one skilled in the art. In addition, any feature or combination of features described or referenced may be specifically included, replicated and/or excluded, in any combination, in/from any embodiment of the present invention. For purposes of summarizing the present invention, certain aspects, advantages and novel features of the present invention are described or referenced. Of course, it is to be understood that not necessarily all such aspects, advantages or features will be embodied in any particular implementation of the present invention. Additional advantages and aspects of the present invention are apparent in the following detailed description and claims that follow.

### BRIEF DESCRIPTION OF THE DRAWINGS

FIG. 1 is a schematic view of an electromagnetic radiation emitting toothtool and a container of identifying compound in a package according to an embodiment of the invention; and

FIGS. 2-4 are schematic views of an electromagnetic radiation emitting toothtool according to another embodiment of the invention.

### DETAILED DESCRIPTION OF THE PRESENT INVENTION

Embodiments of the invention are now described and illustrated in the accompanying drawings, instances of which are to be interpreted to be to scale in some implementations while in other implementations, for each instance, not. In certain aspects, use of like or the same reference designators in the drawings and description refers to the same, similar or analogous components and/or elements, while according to other implementations the same use should not. According to certain implementations, use of directional terms, such as, top, bottom, left, right, up, down, over, above, below, beneath, rear, and front, are to be construed literally, while in other implementations the same use should not. The present invention may be practiced in conjunction with various devices and techniques that are conventionally used in the art, and only so much of the commonly practiced features or process steps are included herein as are necessary to provide an understanding of the present invention. The present invention has applicability in the field of medical and oral-tissue illuminating devices and processes in general. For illustrative purposes, however, the following description pertains to a device for disclosing undesired-substances.

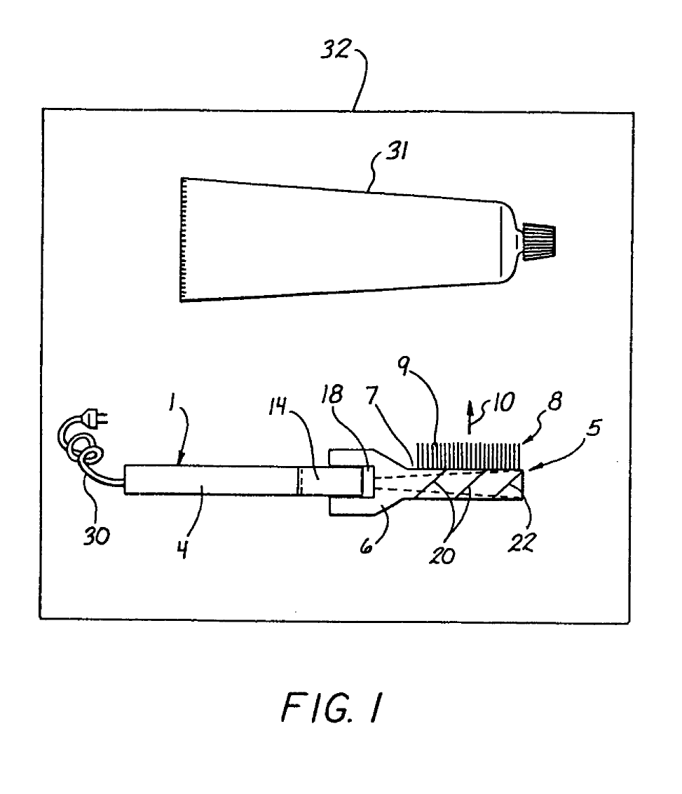

Referring now to FIG. 1, an electromagnetic radiation emitting hand-held device (hereinafter, toothtool or toothbrush although not limited to bristles or even a cleaning surface) 1 and a container 31 of disclosing compound are shown disposed within a package 32. Other embodiments of the invention can have differing features and designs in relation to the toothtool and container 31. The package 32 is shown schematically, and particular embodiments of the invention may use any suitable package design and configuration.

The general structure of the toothtool 1 may, in one embodiment, correspond to that of U.S. Pat. No. 5,306,143 entitled DENTAL HYGIENE APPLIANCE to Levy, the contents of which are incorporated herein by reference. Modified embodiments of the invention may use other suitable electromagnetic radiation emitting toothtools. The basic components of the toothtool 1 may include a handle 4 and a disclosing head 5. The disclosing head 5 is constituted by a body 6 having a target-facing surface 7 from which an emitting structure such as one comprising lightguides and/or a cleaning surface such as a set of tooth bristles 8 may be disposed. The composition and form of the structures 8, and the manner in which the structures 8 are secured to the body 6, can conform to conventional practice in the art. The ends 9 of the structures 8 can comprise energy emitting and/or receiving structures and/or a cleaning surface for scouring teeth and alike. Other embodiments of the invention may have other types of target-facing surfaces, such as, for example, an oraficed surface emitting light therethrough, a sponge or other type of foam.

The toothtool 1 is further provided with means for directing radiation (e.g., polychromatic radiation) of a selected type from the body 6 in the direction of an arrow 10, which is generally parallel to the direction in which the structures 8 may project from the body 6. The radiation is emitted generally via the base surface 7 of the body 6. In modified embodiments of the invention, the radiation may be emitted from the handle 4, guided by the structures 8, or any other suitable means for directing radiation to the ends 9 of the structures, which form the target-facing (e.g., cleaning) surface of the illustrated embodiment of the invention.

The handle 4 is provided with a light-emitting device 14 for generating the electromagnetic radiation. Embodiments of the invention may use any suitable means for generating the electromagnetic radiation, such a semiconductor laser that generates monochromatic electromagnetic radiation or a light emitting diode that emits polychromatic electromagnetic radiation. The light emitting diode can be configured to emit, for example, blue polychromatic electromagnetic radiation. In other implementations, the light emitting diode can be configured to emit, for example, one or more of blue, green, green-violet, blue green and blue violet polychromatic electromagnetic radiation.

The disclosing head 5 is shown having a lens 18, one or more semitransparent mirrors 20 and a fully reflecting mirror 22. In the illustrated embodiment of the invention, the electromagnetic radiation produced by light-emitting device 14 is in the form of a small diameter collimated beam extended along an axis corresponding to the longitudinal axis of the handle 4 and the head 5. The mirrors 20 and 22 are oriented at an angle of 45 degrees to the beam axis, and the lens 18 is constructed and arranged to give the electromagnetic radiation beam a slightly diverging form such that the beam will diverge to an area essentially coextensive with the area occupied by the mirror 22. Modified embodiments of the invention may use other arrangements for directing the electromagnetic radiation generating means to the target-facing (e.g., cleaning) surface of the disclosing head 5.

The body 6 may itself be made of a plastic which is transparent to the electromagnetic radiation so that portions of the radiation are reflected in the direction 10 by the mirrors 20, and the remaining radiation is reflected by the mirror 22. The optical system constituted by the lens 18 and the mirrors 20, 22 is arranged to cause radiation to traverse an area, which is at least approximately coextensive with the area covered by the ends 9 of the structures 8. However, the radiation area may, depending on the particular operations to be performed, extend over a smaller or larger target-facing (e.g., cleaning) surface. Modified embodiments of the invention may use other optical system arrangements.

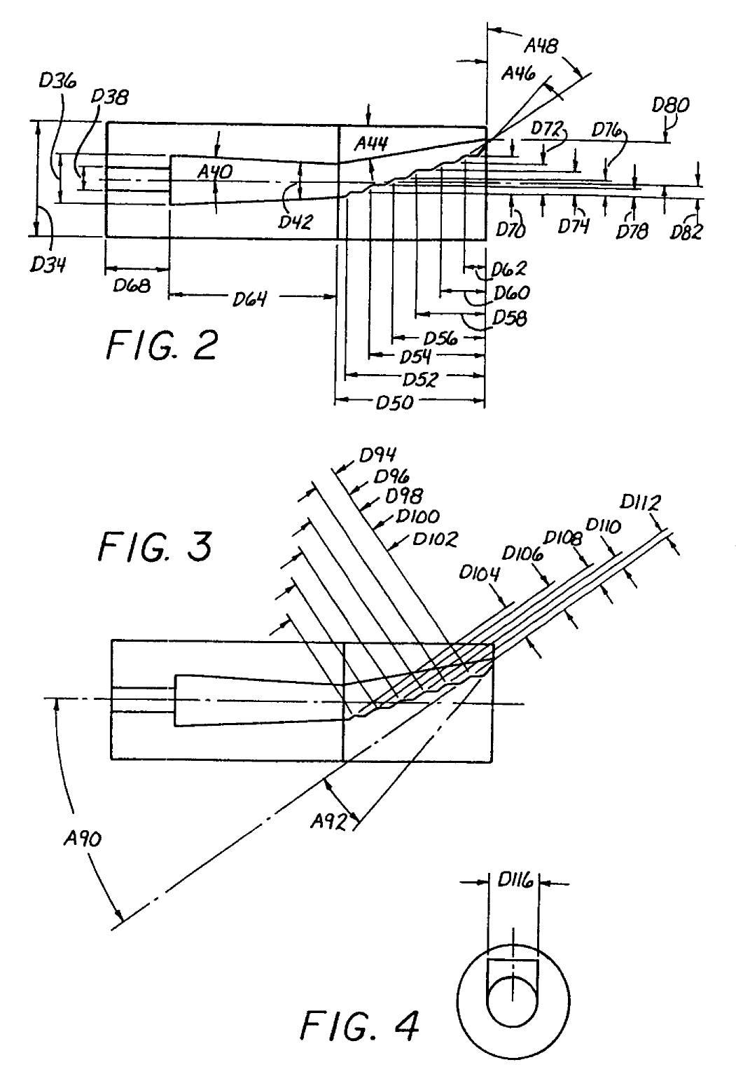

A modified embodiment is illustrated in FIGS. 2-4. The mirrors 20, 22 are replaced with reflective surfaces. The reflective surfaces can be formed on the interior surfaces of the outer, stepped portion of the head of the toothtool. The reflective surfaces may comprise foil or foils, for example, which are embedded into the plastic and wrapped, for example, around the back and sides of the stepped portion of the head. Other materials and/or types of reflective surfaces may be used to increase, decrease, and/or change a distribution of radiation transmitted through the structures.

In FIG. 2, the head tapers from a diameter of 0.440 units, to a diameter of 0.287 units just before the stepped portion. This diameter, which is just before the stepped portion, can be increased to facilitate greater transmission of radiation to the stepped portion or, in other embodiments, can be decreased to attenuate an amount of radiation transmitted to the stepped portion. A width of the head, which is 0.430 units as shown in FIG. 4, may similarly be increased to facilitate greater transmission of radiation through the bristles/structures or, in other embodiments, may be decreased to attenuate an amount of radiation transmitted through the structure. The number, angles, sizes, etc. of the steps forming the reflective surfaces may be changed to increase, decrease, and/or change a distribution of radiation through the structures.

In other embodiments of the invention prior art toothbrushes equipped with a light source, which illuminates the target region and which provides beneficial radiation to the tooth surface, can be used. Examples of illuminating toothbrushes are disclosed in U.S. Pat. Nos. 5,306,143 entitled DENTAL HYGIENE APPLIANCE; 5,160,194 entitled TOOTHBRUSH WITH EXTERNALLY ILLUMINATED BRISTLES; 5,030,090 entitled OPTICAL TOOTHBRUSH AND METHOD OF USE; and 4,779,173 entitled ILLUMINATED BRUSH DEVICE, all of which are expressed incorporated herein by reference in their entireties.

The handle 4 may include a power cord 30 which may be plugged into a wall outlet in order to supply operating power to the light-emitting device 14. Other embodiments of the invention may have a replaceable or rechargeable battery in the handle 4 as the power source for the light-emitting device 14. In addition, in the illustrated embodiment, disclosing head 5 is readily detachable from handle 4. This allows for a plurality of disclosing heads 5, each used by a different individual, to be mounted on handle 4 for use. Additionally, lens 18 may form a unit with handle 4, so that each of the disclosing heads 5 need not be provided with its own lens. This configuration would reduce the cost associated with manufacturing each head 5. Modified embodiments of the invention may have the electromagnetic radiation supplied from a source external to the toothtool 1 via a fiber-optic cable, as is known to those skilled in the art.

In one embodiment, the toothtool 1 emits polychromatic electromagnetic radiation of a wavelength and an intensity for facilitating identification of undesired substances of/on a target surface. Typical undesired substances include bacteria, plaque, tartar, and calculus, which may contribute to, or are precursors of, tooth decay. In other embodiments of the invention monochromatic electromagnetic radiation is emitted to accomplish similar objectives.

The dentifrice is formulated in one embodiment to comprise one or more photosensitive agents (e.g., dyes) that assist in identifying undesired substances on teeth, such as stains, bacteria, plaque, tartar, and calculus. The photosensitive agent(s) react to the emitted electromagnetic radiation of the toothtool during use to enhance identification of the undesired substances.

Other embodiments of the invention comprise dentifrices used in connection with a photosensitive agent comprising a foaming agent. The foaming agent generates foam in the presence of the electromagnetic radiation. The foam operates as a carrier that delivers dentifrice to areas not reachable by the target-facing surface.

When certain photosensitive agents are brought into contact with one or more of the undesired substances, the application of electromagnetic radiation having an appropriate wavelength and energy content can render the substance or substances visible.

By way of example, one of the purposes of tooth brushing is to remove bacteria which have collected on tooth surfaces, frequently in pits and fissures in the tooth enamel. It is known that regardless of the care exercised and time spent in a brushing session, some of the bacteria deposits may not be dislodged and removed from the tooth surfaces. It is also known that there are chemical products, known as disclosing solutions, which can make any deposited bacteria visible. There are other photosensitive agents, which may be preferable to the conventional disclosing solutions, which will react with (e.g., adhere to) bacteria in such a manner as to render the bacteria visible in the presence of radiation having a certain wavelength or wavelengths.

When such a dentifrice is employed, the photosensitive agent comprises a disclosing agent that renders any existing bacteria visible under the radiation produced by the light-emitting device 14 by changing or enhancing the color or appearance of the disclosing agent. The disclosing agent can have an affinity for one or more of the undesired substances, such as bacteria and/or plaque, resulting in the disclosing agent being concentrated about the undesired substances relative to other areas on the target surface. As a result, the radiated, disclosing agent announces locations of undesired substances on tooth surfaces. Yellow and/or orange-colored dentifrices of embodiments of the invention may visually announce undesired substances (e.g., when illuminated with blue light or, in modified embodiments, with one or more of a blue, green, green-violet, blue-green and blue-violet light) during brushing as well with a fluorescing green (or, in a modified embodiment, and/or yellow, or, in a modified embodiment, and/or blue) color. The dentifrices of other embodiments may visually announce undesired substances with a fluorescing green and/or yellow (or, in a modified embodiment, and/or blue, and/or any combination thereof) color. Additional or alternative implementations may comprise a dentifrice with: an agent/dye reactive or highly or most reactive to (e.g., having an isosbestic point in the color) violet, blue and/or green stimulation light to cause caries to fluoresce blue, green, and/or yellow, (e.g., respectively); the caries fluorescing blue (e.g., rather than green); the caries fluorescing blue-green (e.g., rather than just green); and/or a dentifrice with an agent/dye reactive or highly or most reactive to (e.g., having an isosbestic point in the color) blue, green, and/or yellow stimulation light to cause caries to fluoresce green, yellow, and/or orange, (e.g., respectively).

According to one exemplary embodiment of the invention, the chemical product and/or particles consists of or contains an orange, yellow and/or orange-yellow dye or color, such as, for example, one or more of fluorescein, the color additive D&C Yellow no. 7, dibromofluorescein, and/or a disodium salt form of fluorescein (e.g., D&C Yellow no. 8). Salt forms of other agents/dyes may alternatively or additionally be used in the dentifrice and/or particles of the dentifrice. Typically, the dentifrice will be an orange (e.g., and/or yellow) paste with no peroxide, with silica as an abrasive compound for removing stains, and/or with fluoride or a fluoride containing compound.

In an embodiment with the dye being fluorescein, the radiation source can be configured to produce radiation at a wavelength, or wavelengths, containing at least a wavelength of about 494 nm. For instance, the light emitted can comprise one or more of blue light centered at or around 475 nm and green light centered at or around 510 nm. In one implementation, a spectrum of wavelengths spanning about 470 nm to about 520 nm may be emitted by the toothtool. Typically, blue light is embodied to the exclusion of green for reasons including an ability of a blue LED to emit blue light at one or more of a greater efficiency and a higher intensity (e.g., greater brightness) relative to an LED emitting one or more of green, violet, blue-green, blue-violet, green-violet, combinations thereof, and combinations thereof with any other wavelength or wavelengths. In a preferred embodiment, blue light is embodied to the exclusion of green and violet for reasons including an ability of a blue LED to emit blue light at one or more of a greater efficiency and a higher intensity (e.g., greater brightness) relative to an LED emitting one or more of green, violet, blue-green, blue-violet, green-violet, combinations thereof, and combinations thereof with any other wavelength or wavelengths.

The toothtool may, for example, emit electromagnetic radiation wavelengths consisting essentially of non-ultraviolet radiation; may emit electromagnetic radiation consisting essentially of wavelengths bound within a range of about 300 to 750 nanometers; emit electromagnetic radiation with a peak power at one or more of about blue, green, green-violet, blue-green and blue-violet wavelengths; and/or emit visible-light excluding a visible wavelength that is not blue, not green, not green-violet, not blue-green and not blue-violet. Furthermore, the toothtool may, in other examples, additionally or alternatively comprise a configuration that filters, attenuates, or blocks electromagnetic radiation with a visible-light wavelength that is outside of a blue, green, blue-green of blue-violet wavelength; filters, attenuates, or blocks a non-blue wavelength; filters, attenuates, or blocks a visible-light wavelength corresponding to light that is neither blue, green, nor violet; emits electromagnetic radiation wavelengths consisting essentially of non-ultraviolet radiation; emits visible-light excluding a wavelength beyond blue, green, greenviolet, bluegreen or blueviolet; emits more of a first band of various visible-light wavelengths than a second band of various visible-light wavelengths, the second band of various visible-light wavelengths excluding one or more of blue, green, green-violet, blue-green and blue-violet; filters, attenuates, or blocks a non-blue wavelength; and/or emits more of a first band of various visible-light wavelengths than a second band of various visible-light wavelengths, the second band of various visible-light wavelengths excluding blue.

Agents comprising orange, supplied, for example, by Crompton & Knowles, Ingredient Technology Division, of Reading, Pa., may be selected, along with radiation source(s) having corresponding wavelengths. Agents comprising yellows, supplied, for example, by Crompton & Knowles, Ingredient Technology Division, may alternatively/additionally be selected, along with radiation source(s) having corresponding wavelengths. Also, agents comprising reds, browns, greens, and/or blues, etc., supplied, for example, by Crompton & Knowles, Ingredient Technology Division, may alternatively/additionally be selected, along with radiation source(s) having corresponding wavelengths. In an embodiment of the invention, an optimum amount of transmission through the dentifrice comprises transmitting electromagnetic radiation at wavelengths and intensities thereof to facilitate reaction of the photosensitive agent, and substantially no additional electromagnetic radiation beyond that. Embodiments of the invention can have the photosensitive agents dispersed throughout the dentifrice.

In addition to engineering the dentifrice to remain stable until placed on the tooth and agitated, a method of the invention comprises a step of instructing the user to leave the source of the toothtool in an off mode until the toothtool and dentifrice are placed on the teeth. The user follows the instructions and inserts the toothtool, with the dentifrice thereon and with the source in an off mode, into the mouth and onto the teeth of the user. Subsequently, the user places the source into an on mode to thereby initiate the emission of radiation from the toothtool into the dentifrice. The user can then move the bristles/structures of the toothtool on the teeth to agitate and further activate the active ingredient or ingredients of the dentifrice. In selected embodiments, the mere placement of the toothtool and/or dentifrice on or near a tooth, with or without agitation, initiates activation of the ingredient or ingredients of the dentifrice.

In other embodiments of the invention, the dentifrice may comprise abrasives. The abrasives may be visible, an example of which is disclosed in U.S. Pat. No. 3,935,306 entitled TOOTHPASTE FORMULATIONS, which is incorporated herein by reference. The abrasives may be clear, an example of which is disclosed in U.S. Pat. No. 3,864,470 entitled VISUALLY CLEAR TOOTHPASTE CONTAINING SYNTHETIC PRECIPITATED HYDRATED SILICA, which is incorporated herein by reference. Clear abrasive particles enhance the transmissibility of the dentifrice, as compared to opaque abrasive particles.

In an embodiment of the invention, the electromagnetic radiation emitted from the toothtool 1 is substantially free of ultraviolet radiation. Ultraviolet radiation is a relatively high energy wavelength range, compared to visible and infrared wavelengths. Under some circumstances, directing ultraviolet radiation into the mouth may result in cellular damage. Further, as the ultraviolet radiation is higher energy, the toothtool 1 may consume less energy during operation as it does not emit the higher energy ultraviolet wavelengths.

In an embodiment of the invention, a toothtool emits electromagnetic radiation wavelengths consisting essentially of non-ultraviolet radiation, or, consisting essentially of wavelengths within a range of 300 to 750 nanometers, with a peak power of blue light, and/or with wavelengths only in or with added power in the blue region or in one or more of blue, green, green violet, blue green and blue violet wavelengths.

One embodiment of the invention emits electromagnetic radiation wavelengths consisting essentially of non-ultraviolet radiation, or, consisting essentially of wavelengths within a range of 300 to 750 nanometers, with a peak power of or in a vicinity of one or more of blue, green, green violet, blue green and blue violet wavelengths. Another embodiment of the invention emits electromagnetic radiation wavelengths consisting essentially of non-ultraviolet radiation, or, consisting essentially of wavelengths within a range of 300 to 750 nanometers, with visible-light of wavelengths excluding one or more of blue, green, green violet, blue green and blue violet, being filtered, attenuated, or blocked. Other embodiments of the invention comprise a toothtool that emits electromagnetic radiation within the infrared or near-infrared region, with a peak power of or in a vicinity of blue light, and/or with wavelengths only in or with added power in the blue region or in one or more of blue, green, green violet, blue green and blue violet wavelengths. Compounds can be selected to react with this light exclusively or in addition to other wavelengths.

By way of the disclosure herein, a wand which may comprise a laser assembly is described that can output radiation useful to diagnose, monitor and/or affect a target surface. For example, embodiments of the invention may have a toothtool emitting electromagnetic radiation which may be coherent or non-coherent.

According to certain implementations, energy from the device (e.g., wand, probe and/or handpiece) is directed, for example, into fluid (e.g., an air and/or water spray or an atomized distribution of fluid particles from a water connection and/or a spray connection near an output end of a handpiece) that is emitted from a fluid output of a handpiece above a target surface (e.g., one or more of tooth, bone, cartilage and soft tissue). The fluid output may comprise a plurality of fluid outputs, concentrically arranged around a power fiber, as described in, for example, application Ser. No. 11/042,824 and Prov. App. 60/601,415. The power or treatment fiber may be coupled to an energy source comprising one or more of a wavelength within a range from about 2.69 to about 2.80 microns and a wavelength of about 2.94 microns. In certain implementations the power fiber may be coupled to one or more of an Er:YAG laser, an Er:YSGG laser, an Er, Cr:YSGG laser and a CTE:YAG laser, and in particular instances may be coupled to one of an Er, Cr:YSGG solid state laser having a wavelength of about 2.789 microns and an Er:YAG solid state laser having a wavelength of about 2.940 microns. An apparatus including corresponding structure for directing energy into an atomized distribution of fluid particles above a target surface is disclosed, for example, in the below-referenced U.S. Pat. No. 5,574,247, which describes the impartation of laser energy into fluid particles to thereby apply disruptive forces to the target surface.

In the case of procedures using fiber optic tip radiation, a probe can include one or more power or treatment fibers for transmitting treatment radiation to a target surface for treating (e.g., ablating) a dental structure, such as within a canal. In any of the embodiments described herein, the light for illumination and/or diagnostics may be transmitted simultaneously with, or intermittently with or separate from, transmission of treatment radiation and/or of the fluid from the fluid output or outputs.

Additional aspects, implementations, and embodiments of the invention are enumerated as follows: (1) toothbrush that is brush-less and emits light to shine on teeth together with a rinse to show undesired substances; (2) no brush/bristles needed, emit light onto teeth together with a disclosing substance; e.g., give luminescence (changing color) and/or fluorescence; (3) can have two lights: one wavelength does not transmit or transmits less through the undesired substance (e.g., plaque) and the other is more transmissive through the undesired substance so may detect thickness of the undesired substance; as for a disclosing agent implementation, can have two lights: one wavelength does not transmit or transmits less through the undesired substance (e.g., plaque), as colored/treated by disclosing agent, and the other is more transmissive through the undesired substance (e.g., plaque), as colored/treated by disclosing agent, so may detect thickness of the undesired substance; (4) in one embodiment the device light turns on upon detecting a certain wavelength/color from the tooth (e.g., with a magnitude above a threshold in, e.g., the fluorescing feedback) indicative, e.g., of the presence of an undesired substance (e.g., plaque) while, e.g., brushing, also can have device light turn off when no longer detected (e.g., under threshold); (5) can have two disclosing agents: one is transparent and one not, or one is the reference (so will provide reference for change of color . . . ), emitting surface of device can have different heights like protruding rubber tips that deliver light; (6) or could have rubber conduits protruding (e.g., through brushes) for delivery of light into tooth areas may have different flexibility (some stiffer).

Corresponding or related structure and methods described in the following patents assigned to Biolase Technology, Inc. are incorporated herein by reference in their entireties, wherein such incorporation includes corresponding or related structure (and modifications thereof) in the following patents which may be, in whole or in part, (i) operable with, (ii) modified by one skilled in the art to be operable with, and/or (iii) implemented/used with or in combination with, any part(s) of the present invention according to this disclosure, that of the patents or below applications, and the knowledge and judgment of one skilled in the art.

Such patents include, but are not limited to U.S. Pat. No. 7,578,622 entitled Contra-angle rotating handpiece having tactile-feedback tip ferrule; U.S. Pat. No. 7,575,381 entitled Fiber tip detector apparatus and related methods; U.S. Pat. No. 7,563,226 entitled Handpieces having illumination and laser outputs; U.S. Pat. No. 7,467,946 entitled Electromagnetic radiation emitting device and dentifrice system; U.S. Pat. No. 7,461,982 entitled Contra-angle rotating handpiece having tactile-feedback tip ferrule; U.S. Pat. No. 7,461,658 entitled Methods for treating eye conditions; U.S. Pat. No. 7,458,380 entitled Methods for treating eye conditions; U.S. Pat. No. 7,424,199 entitled Fiber tip fluid output device; U.S. Pat. No. 7,421,186 entitled Modified-output fiber optic tips; U.S. Pat. No. 7,415,050 entitled Electromagnetic energy distributions for electromagnetically induced mechanical cutting; U.S. Pat. No. 7,384,419 entitled Tapered fused waveguide for delivering treatment radiation toward a target surface; U.S. Pat. No. 7,356,208 entitled Fiber detector apparatus and related methods; U.S. Pat. No. 7,320,594 entitled Fluid and laser system; U.S. Pat. No. 7,303,397 entitled Caries detection using timing differentials between excitation and return pulses; U.S. Pat. No. 7,292,759 entitled Contra-angle rotating handpiece having tactile-feedback tip ferrule; U.S. Pat. No. 7,290,940 entitled Fiber tip detector apparatus and related methods; U.S. Pat. No. 7,288,086 entitled High-efficiency, side-pumped diode laser system; U.S. Pat. No. 7,270,657 entitled Radiation emitting apparatus with spatially controllable output energy distributions; U.S. Pat. No. 7,261,558 entitled Electromagnetic radiation emitting device and dentifrice system; U.S. Pat. No. 7,194,180 entitled Fiber detector apparatus and related methods; U.S. Pat. No. 7,187,822 entitled Fiber tip fluid output device; U.S. Pat. No. 7,144,249 entitled Device for dental care and whitening; U.S. Pat. No. 7,108,693 entitled Electromagnetic energy distributions for electromagnetically induced mechanical cutting; U.S. Pat. No. 7,068,912 entitled Fiber detector apparatus and related methods; U.S. Pat. No. 6,942,658 entitled Radiation emitting apparatus with spatially controllable output energy distributions; U.S. Pat. No. 6,829,427 entitled Fiber detector apparatus and related methods; U.S. Pat. No. 6,821,272 entitled Electromagnetic energy distributions for electromagnetically induced cutting; U.S. Pat. No. 6,744,790 entitled Device for reduction of thermal lensing; U.S. Pat. No. 6,669,685 entitled Tissue remover and method; U.S. Pat. No. 6,616,451 entitled Electromagnetic radiation emitting device and dentifrice system; U.S. Pat. No. 6,616,447 entitled Device for dental care and whitening; U.S. Pat. No. 6,610,053 entitled Methods of using atomized particles for electromagnetically induced cutting; U.S. Pat. No. 6,567,582 entitled Fiber tip fluid output device; U.S. Pat. No. 6,561,803 entitled Fluid conditioning system; U.S. Pat. No. 6,544,256 entitled Electromagnetically induced cutting with atomized fluid particles for dermatological applications; U.S. Pat. No. 6,533,775 entitled Light-activated hair treatment and removal device; U.S. Pat. No. 6,389,193 entitled Rotating handpiece; U.S. Pat. No. 6,350,123 entitled Fluid conditioning system; U.S. Pat. No. 6,288,499 entitled Electromagnetic energy distributions for electromagnetically induced mechanical cutting; U.S. Pat. No. 6,254,597 entitled Tissue remover and method; U.S. Pat. No. 6,231,567 entitled Material remover and method; U.S. Pat. No. 6,086,367 entitled Dental and medical procedures employing laser radiation; U.S. Pat. No. 5,968,037 entitled User programmable combination of atomized particles for electromagnetically induced cutting; U.S. Pat. No. 5,785,521 entitled Fluid conditioning system; U.S. Pat. No. 5,741,247 entitled Atomized fluid particles for electromagnetically induced cutting; and the following patents identified by number only: U.S. Pat. No. 7,167,752, U.S. Pat. No. 6,494,900, U.S. Pat. No. 5,995,873, U.S. Pat. No. 5,674,261, U.S. Pat. No. 5,578,060, U.S. Pat. No. 5,024,236 and U.S. Pat. No. 4,535,784.

Also, the above disclosure and referenced items, and that described on the referenced pages, are intended to be operable or modifiable to be operable, in whole or in part, with corresponding or related structure and methods, in whole or in part, described in the following published applications and items referenced therein, which applications are listed as follows: App. Pub. 20090225060 entitled Wrist-mounted laser with animated, page-based graphical user-interface; App. Pub. 20090143775 entitled Medical laser having controlled-temperature and sterilized fluid output; App. Pub. 20090141752 entitled Dual pulse-width medical laser with presets; App. Pub. 20090105707 entitled Drill and flavored fluid particles combination; App. Pub. 20090104580 entitled Fluid and pulsed energy output system; App. Pub. 20090076490 entitled Fiber tip fluid output device; App. Pub. 20090075229 entitled Probes and biofluids for treating and removing deposits from tissue surfaces; App. Pub. 20090067189 entitled Contra-angle rotating handpiece having tactile-feedback tip ferrule; App. Pub. 20090062779 entitled Methods for treating eye conditions with low-level light therapy; App. Pub. 20090056044 entitled Electromagnetic radiation emitting device and dentifrice system; App. Pub. 20090043364 entitled Electromagnetic energy distributions for Electromagnetically induced mechanical cutting; App. Pub. 20090042171 entitled Fluid controllable laser endodontic cleaning and disinfecting system; App. Pub. 20090035717 entitled Electromagnetic radiation emitting device and transparent dentifrice system; App. Pub. 20090031515 entitled Transparent dentifrice for use with radiation emitting device system; App. Pub. 20080317429 entitled Modified-output fiber optic tips; App. Pub. 20080276192 entitled Method and apparatus for controlling an energy output system; App. Pub. 20080240172 entitled Radiation emitting apparatus with spatially controllable output energy distributions; App. Pub. 20080221558 entitled Multiple fiber-type tissue treatment device and related method; App. Pub. 20080219629 entitled Modified-output fiber optic tips; App. Pub. 20080212624 entitled Dual pulse-width medical laser; App. Pub. 20080203280 entitled Target-close energy emitting device; App. Pub. 20080181278 entitled Electromagnetic energy output system; App. Pub. 20080181261 entitled Electromagnetic energy output system; App. Pub. 20080157690 entitled Electromagnetic energy distributions for electromagnetically induced mechanical cutting; App. Pub. 20080151953 entitled Electromagnet energy distributions for electromagnetically induced mechanical cutting; App. Pub. 20080138764 entitled Fluid and laser system; App. Pub. 20080125677 entitled Methods for treating hyperopia and presbyopia via laser tunneling; App. Pub. 20080125676 entitled Methods for treating hyperopia and presbyopia via laser tunneling; App. Pub. 20080097418 entitled Methods for treating eye conditions; App. Pub. 20080097417 entitled Methods for treating eye conditions; App. Pub. 20080097416 entitled Methods for treating eye conditions; App. Pub. 20080070185 entitled Caries detection using timing differentials between excitation and return pulses; App. Pub. 20080069172 entitled Electromagnetic energy distributions for electromagnetically induced mechanical cutting; App. Pub. 20080065057 entitled High-efficiency, side-pumped diode laser system; App. Pub. 20080065055 entitled Methods for treating eye conditions; App. Pub. 20080065054 entitled Methods for treating hyperopia and presbyopia via laser tunneling; App. Pub. 20080065053 entitled Methods for treating eye conditions; App. Pub. 20080033411 entitled High efficiency laser energy cutting device; App. Pub. 20080033409 entitled Methods for treating eye conditions; App. Pub. 20080033407 entitled Methods for treating eye conditions; App. Pub. 20080025675 entitled Fiber tip detector apparatus and related methods; App. Pub. 20080025672 entitled Contra-angle rotating handpiece having tactile-feedback tip ferrule; App. Pub. 20080025671 entitled Contra-angle rotating handpiece having tactile-feedback tip ferrule; App. Pub. 20070298369 entitled Electromagnetic radiation emitting device and dentifrice system; App. Pub. 20070263975 entitled Modified-output fiber optic tips; App. Pub. 20070258693 entitled Fiber detector apparatus and related methods; App. Pub. 20070208404 entitled Tissue treatment device and method; App. Pub. 20070208328 entitled Contra-angel rotating handpiece having tactile-feedback tip ferrule; App. Pub. 20070190482 entitled Fluid conditioning system; App. Pub. 20070184402 entitled Caries detection using real-time imaging and multiple excitation frequencies; App. Pub. 20070128576 entitled Output attachments coded for use with—energy procedural device; App. Pub. 20070104419 entitled Fiber tip fluid output device; App. Pub. 20070060917 entitled High-efficiency, side-pumped diode laser system; App. Pub. 20070059660 entitled Device for dental care and whitening; App. Pub. 20070054236 entitled Device for dental care and whitening; App. Pub. 20070054235 entitled Device for dental care and whitening; App. Pub. 20070054233 entitled Device for dental care and whitening; App. Pub. 20070042315 entitled Visual feedback implements for energy output devices; App. Pub. 20070016176 entitled Laser handpiece architecture and methods; App. Pub. 20070014517 entitled Electromagnetic energy emitting device with increased spot size; App. Pub. 20070014322 entitled Electromagnetic energy distributions for electromagnetically induced mechanical cutting; App. Pub. 20070009856 entitled Device having activated textured surfaces for treating oral tissue; App. Pub. 20070003604 entitled Tissue coverings bearing customized tissue images; App. Pub. 20060281042 entitled Electromagnetic radiation emitting device and dentifrice system; App. Pub. 20060275016 entitled Contra-angle rotating handpiece having tactile-feedback tip ferrule; App. Pub. 20060241574 entitled Electromagnetic energy distributions for electromagnetically induced disruptive cutting; App. Pub. 20060240381 entitled Fluid conditioning system; App. Pub. 20060210228 entitled Fiber detector apparatus and related methods; App. Pub. 20060204203 entitled Radiation emitting apparatus with spatially controllable output energy distributions; App. Pub. 20060142745 entitled Dual pulse-width medical laser with presets; App. Pub. 20060142744 entitled Identification connector for a medical laser handpiece; App. Pub. 20060142743 entitled Medical laser having controlled-temperature and sterilized fluid output; App. Pub. 20060126680 entitled Dual pulse-width medical laser; App. Pub. 20060099548 entitled Caries detection using timing differentials between excitation and return pulses; App. Pub. 20060083466 entitled Fiber tip detector apparatus and related methods; App. Pub. 20060043903 entitled Electromagnetic energy distributions for electromagnetically induced mechanical cutting; App. Pub. 20050283143 entitled Tissue remover and method; App. Pub. 20050281887 entitled Fluid conditioning system; App. Pub. 20050281530 entitled Modified-output fiber optic tips; App. Pub. 20050256517 entitled Electromagnetically induced treatment devices and methods; App. Pub. 20050256516 entitled Illumination device and related methods; App. Pub. 20040106082 entitled Device for dental care and whitening; App. Pub. 20040092925 entitled Methods of using atomized particles for electromagnetically induced cutting; App. Pub. 20040091834 entitled Electromagnetic radiation emitting device and dentifrice system; App. Pub. 20040068256 entitled Tissue remover and method; App. Pub. 20030228094 entitled Fiber tip fluid output device; App. Pub. 20020149324 entitled Electromagnetic energy distributions for electromagnetically induced mechanical cutting; App. Pub. 20020014855 entitled Electromagnetic energy distributions for electromagnetically induced mechanical cutting; the following patent application publications identified by number only: 20080097564, 2006019564 and 20030208245; and the document entitled Hand held scanner and the document labeled BI9485P both of which are included in the above-referenced U.S. Provisional Application No. 61/287,497.

Subject matter from the Hand held scanner and BI9485P documents is reproduced in this and the following paragraph. One feature can be to maintain a bounded layer of fluid particles, which is not too thick and which is not too thin. A fiber optic tip (e.g., the material tip and/or the emitting end) placed into (e.g., and/or placing treatment energy into) a distribution of fluid particles and, additionally, placed in close proximity (2-3 mm, for example) of a target surface, creates a thin layer of fluid particles between the incident, concentrated electromagnetic energy and the target surface. Other distances are possible within the scope of the present invention, depending on, for example, the selected laser intensity and wavelength, the selected fluid, and the selected distribution of atomized fluid particles. The below embodiments disclose, for example, other means for creating a bounded layer of fluid particles between the incident, concentrated electromagnetic energy and the target surface. An electromagnetically induced cutter can comprise a laser, microprocessor and user interface. The electromagnetically induced cutter 121 further can comprise an air and/or water source for supplying one or more atomization nozzles with air and/or water. In accordance with an embodiment, one material tips (e.g., contacting arms) may be used, taking on basically any form so long as the one or more contacting arms provide a function of spacing the source of electromagnetic energy from the target surface. For example, in one modified embodiment, the one or more contacting arms may be constructed to contact another surface, such as another part of the patient besides the actual target or a peripheral part of the target, while still providing the function of spacing the source of electromagnetic energy from the target surface. In other modified embodiments, one or more additional tissue contacting arms may be implemented besides just one. For example, three or more tissue contacting arms may be disposed at, for example, about 120 degrees, 240 degrees and 360 degrees. In another embodiment, the tissue contacting arm or arms are part of and form at least a partial enclosure, such as a hemispherical enclosure. In yet another embodiment, the tissue contacting arms form at least a partial cylindrical, rectangular or other enclosure. The contacting surface of the enclosure (i.e., the surface that contacts the target surface) may thus comprise one or more points for actually touching the target surface (corresponding to one or more contacting legs), or may comprise a circular, oval, rectangular or other continuous or non-continuous perimeter for actually touching the target surface. For example, the contacting arms may form an oval, hemispherical enclosure, such as that of an upside down spoon, wherein the contacting surface of the oval, hemispherical enclosure forms an oval shape or edge for touching the target surface. Thus, in use, an oval shape on the target surface would be enclosed by the oval, hemispherical configuration. As used herein, the term “hemispherical” is not intended to define half of a sphere but, rather, to define any closed surface with an opening for contacting the target surface. Thus, in an embodiment wherein the hemispherical configuration forms a rectangular edge for contacting the target surface, the enclosure may have any of a variety of shapes such as for example half or a sphere that transitions into the rectangular edge, or an open ended cubical enclosure with the rectangular edge. The distal ends of the tissue contacting arms are preferably rounded or smooth-surfaced to allow the tissue contacting arms to glide over the target surface, such as a patient's skin, tissue, crystal or glass. In one modified embodiment, at least one of the distal ends comprises a ball roller.

A moisture output directs moist air and/or water or an atomized air/water spray into the path of the electromagnetic energy from the parabolic mirror or prism. Water from the moisture output can help to allow the tissue contacting arms to slide over the target surface. In one embodiment, water or another fluid, or an additive to water, having lubricating properties, may be emitted from the moisture output. For example, soft water may be emitted from the moisture output. As presently preferred, the moisture output comprises an atomizer for outputting atomized fluid particles into the path of the electromagnetic energy above or on the target surface, and the parabolic mirror or prism 188 focuses the electromagnetic energy into an interaction zone above, on or within (interstitially) the target surface. A suction removes excess moist air and/or atomized fluid particles. The suction is preferably disposed opposite to the moisture output to facilitate a fluid flow path from the moisture output 190, through the interaction zone, and out through the suction. The tissue contacting arms may be formed of stainless steel or a plastic, for example. Part or all of the tissue contacting arms may be formed of a transparent material, such as a transparent plastic. At least one of the tissue contacting arms can comprise a proximal end, a distal end, and a suction passageway extending therebetween. Each suction passageway can be constructed to carry surplus fluids and debris from the target surface. In order to facilitate this end, one or more of the rounded surfaces (e.g., ball rollers) at the distal ends may be configured to have a smaller or flatter profile to place the relative position(s) of the suction passageway opening(s) closer to the target surface. In one embodiment, the opening or openings of the suction passageway(s) may be placed within the rounded surface(s) or ball roller(s) at the distal end(s). Each suction passageway can, for example, remove water particles that have been emitted and carry them proximally through the suction passageway and out of the handpiece. Another suction passageway may be disposed in a second tissue contacting arm(s), with or without additional suction passageway(s). In another embodiment, the tissue contacting arms are part of and form an enclosure, such as a hemispherical enclosure. The distal ends of the tissue contacting arms can be rounded or smooth-surfaced to allow the tissue contacting arms to slide over the target surface, such as a patient's tissue. In a modified embodiment, one or more of the distal ends may comprise a ball roller. Regardless of the shape of the distal end of the tissue contacting arm, water from a moisture output can help the tissue contacting arm or arms glide over the target surface. The air and water lines may be configured to output, soft water or another fluid, or an additive to water, having lubricating properties. One or more atomizers, mist generators, or moist air outputs (fluid outputs) may be disposed in, connected to or fitted between the tissue contacting arms.

All of the contents of the preceding applications, materials, and referenced matters/content are incorporated herein by reference in their entireties. Although the disclosure herein refers to certain illustrated embodiments, it is to be understood that these embodiments have been presented by way of example rather than limitation. For example, any of the radiation/energy outputs (e.g., lasers), any of the fluid outputs (e.g., water outputs), and any conditioning agents, particles, agents, etc., and particulars or features thereof, or other features, including method steps and techniques, may be used with any other structure(s) and process described or referenced herein, in whole or in part, in any combination or permutation as a non-equivalent, separate, non-interchangeable aspect of this invention. Corresponding or related structure and methods specifically contemplated, disclosed, referenced and/or claimed herein as part of this invention, to the extent not mutually inconsistent as will be apparent from the context, this specification, and the knowledge of one skilled in the art, including, modifications thereto, which may be, in whole or in part, (i) operable and/or constructed with, (ii) modified by one skilled in the art to be operable and/or constructed with, and/or (iii) implemented/made/used with or in combination with, any parts of the present invention according to this disclosure, include: (I) any one or more parts of the above disclosed or referenced structure and methods and/or (II) subject matter of any one or more of the following claims and parts thereof, in any permutation and/or combination. For instance, the device/wand can be modified according to any of the above to implement scanning. The intent accompanying this disclosure is to have such embodiments construed in conjunction with the knowledge of one skilled in the art to cover all modifications, variations, combinations, permutations, omissions, substitutions, alternatives, and equivalents of the embodiments, to the extent not mutually exclusive, as may fall within the spirit and scope of the invention.

What is claimed is:

1. An undesirable-substance identifying system for teeth, comprising:
a toothtool having a source of electromagnetic radiation constructed to direct polychromatic electromagnetic radiation toward a target-facing surface, wherein the electromagnetic radiation has a peak power in a vicinity of blue light but is at least partially free of ultraviolet radiation; and
an orange dentifrice comprising a photosensitive agent, which is dispersed throughout the dentifrice, wherein during use the dentifrice is dispersed over a target surface to be absorbed by undesired substances, and fluorescences green when irradiated with the source.

2. The undesirable-substance identifying system of claim 1, wherein the toothtool comprises structures and is constructed to direct polychromatic electromagnetic radiation through the structures toward the target-facing surface.

3. The undesirable-substance identifying system of claim 1, wherein the dentifrice comprises salt and a color of one or more of orange, yellow and orange-yellow.

4. The undesirable-substance identifying system of claim 1, wherein the toothtool comprises structures and is constructed to direct polychromatic electromagnetic radiation around the structures toward the target-facing surface.

5. The undesirable-substance identifying system of claim 1, wherein the source of:
electromagnetic radiation comprises a light emitting diode; and
the dentifrice comprises fluoride and clear abrasive particles.

6. The undesirable-substance identifying system of claim 1, wherein the toothtool emits more of a first band of various visible-light wavelengths than a second band of various visible-light wavelengths, the second band of various visible-light wavelengths excluding one or more of blue, green, green-violet, blue-green and blue-violet.

7. The undesirable-substance identifying system of claim 1, wherein the toothtool emits more of a first band of various visible-light wavelengths than a second band of various visible-light wavelengths, the second band of various visible-light wavelengths excluding blue.

8. The undesirable-substance identifying system of claim 1, wherein the dentifrice comprises one or more of an orange dye, a yellow dye, an orange color, a yellow color, and an orange-yellow color.

9. The undesirable-substance identifying system of claim 8, wherein:
the dentifrice further comprises a salt; and
the toothtool emits electromagnetic radiation wavelengths consisting essentially of non-ultraviolet radiation.

10. The undesirable-substance identifying system of claim 8, wherein the toothtool emits electromagnetic radiation consisting essentially of wavelengths bound within a range of about 300 to 750 nanometers.

11. The undesirable-substance identifying system of claim 8, wherein the toothtool emits electromagnetic radiation with a peak power at one or more of about blue, green, green-violet, blue-green and blue-violet wavelengths.

12. The undesirable-substance identifying system of claim 8, wherein the toothtool emits visible-light excluding a visible wavelength that is not blue, not green, not green-violet, not blue-green and not blue-violet.

13. The undesirable-substance identifying system of claim 8, wherein the toothtool filters, attenuates, or blocks electromagnetic radiation with a visible-light wavelength that is outside of a blue, green, green-violet, blue-green of blue-violet wavelength.

14. The undesirable-substance identifying system of claim 8, wherein the toothtool filters, attenuates, or blocks a non-blue wavelength.

15. The undesirable-substance identifying system of claim 8, wherein the toothtool filters, attenuates, or blocks a visible-light wavelength corresponding to light that is neither blue, green, nor violet.

16. The undesirable-substance identifying system of claim 1, wherein the toothtool emits electromagnetic radiation wavelengths consisting essentially of non-ultraviolet radiation.

17. The undesirable-substance identifying system of claim 1, wherein the toothtool emits electromagnetic radiation consisting essentially of wavelengths bound within a range of about 300 to 750 nanometers.

18. The undesirable-substance identifying system of claim 1, wherein the toothtool emits electromagnetic radiation with a peak power at one or more of blue, green, greenviolet, bluegreen and blueviolet wavelengths.

19. The undesirable-substance identifying system of claim 1, wherein the toothtool emits visible-light excluding a wavelength beyond blue, green, greenviolet, bluegreen or blueviolet.

20. The undesirable-substance identifying system of claim 1, wherein the dentifrice comprises a salt form of an orange dye.
